(19) United States
(12) Plant Patent Application Publication
(10) Pub. No.: US 2008/0184444 P1
(43) Pub. Date: Jul. 31, 2008

(54) BRACHYSCOME PLANT NAMED 'BRAMIPURO'

(75) Inventor: Theodorus Cornelius Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/651,091

(22) Filed: Jan. 8, 2007

(51) Int. Cl. A01H 5/00

(52) U.S. Cl. PLT/411

(57) ABSTRACT

A new Brachyscome multifida plant particularly distinguished by its upright and rounded plant habit, good field performance, freely flowering with numerous inflorescences per plant, and daisy-type inflorescences with purple-violet ray florets.

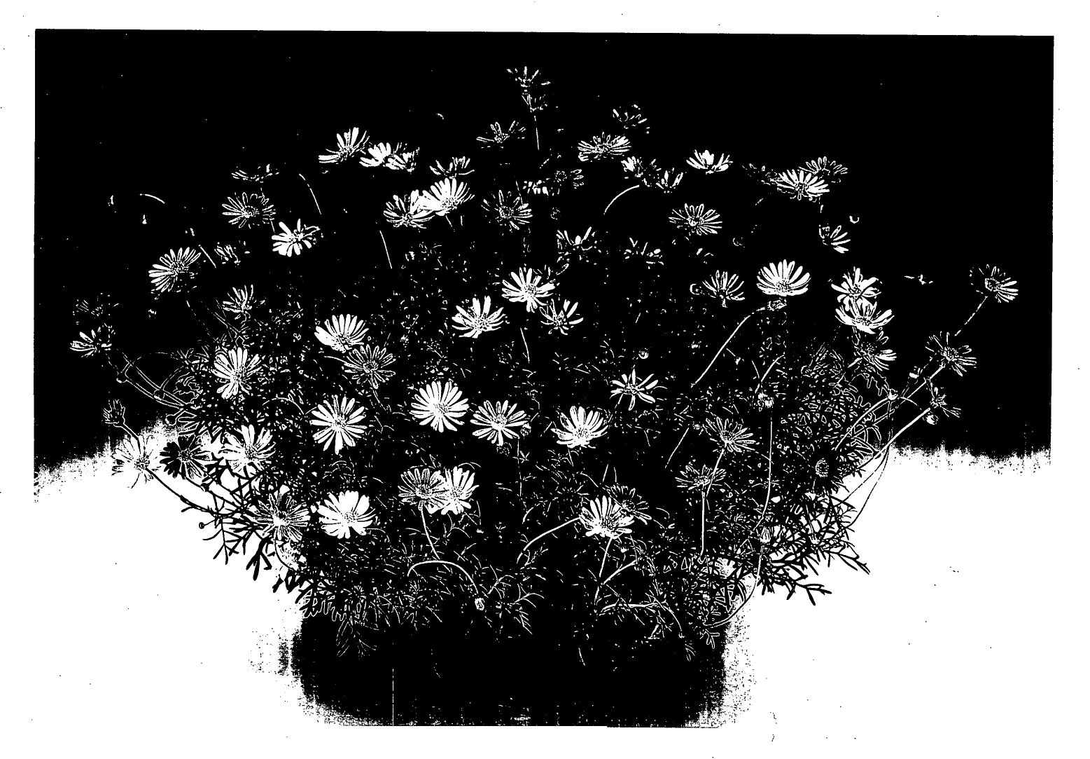

### LATIN NAME OF THE GENUS AND SPECIES OF THE PLANT CLAIMED

Brachyscome multifida

### VARIETAL DENOMINATION

‘Bramipuro’

### BACKGROUND OF THE NEW PLANT

The present invention relates to a new distinct cultivar of Brachyscome, botanically known as Brachyscome multifida.

The new cultivar is propagated by cuttings resulting from an unnamed seedling of Brachyscome multifida no. ‘A317-1.’ Seed of this selection, resulting from open pollination was harvested in 2001 and the new variety ‘Bramipuro’ was selected in 2002 as one of the plants of this population.

Breeder's code of the new plant is ‘D3070-3.’

This new plant is an annual. The new cultivar is a product of a planned breeding program conducted by the inventor in Enkhuizen, The Netherlands. The new cultivar ‘Bramipuro’ is compact and flowers throughout the summer. ‘Bramipuro’ is asexually reproduced by cuttings for 4 years now and trialled under different conditions in Europe. It has retained its distinctive characteristics through successive propagation and is firmly fixed. The new variety reproduces true to type in successive generations of asexual reproduction.

### SUMMARY OF THE INVENTION

The new Brachyscome has not been observed under all possible environmental conditions. The phenotype may vary somewhat with variations in environment such as temperature and light intensity without, however, any variance in genotype.

The following characteristics have been repeatedly observed and are determined to be basic characteristics of ‘Bramipuro.’ These characteristics in combination distinguish ‘Bramipuro’ as a new and distinct cultivar:

1. Compact, upright and rounded plant habit

2. Freely branching habit and short internodes; dense and bushy plant habit.

3. Early flowering

4. Freely flowering habit with numerous small inflorescences per plant.

5. Daisy-type inflorescences with bright pink ray florets.

TABLE 1
DIFFERENCES BETWEEN THE NEW CULTIVAR
‘BRAMIPURO’ AND A SIMILAR VARIETY

| | ‘Bramipuro’ | ‘Blue’ (not patented) |
|---|---|---|
| Plant height | 20 cm | 25 cm |
| Plant width | 42 cm | 60 cm |
| Flower diameter | Up to 15 mm | Up to 20 mm |
| Flower color | RHS N82B | RHS N87B |
| Foliage color | RHS 137A | RHS 143A |

### DESCRIPTION OF THE DRAWING

This new Brachyscome plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Brachyscome plant. The data which define these characteristics were collected from asexual reproductions carried out in Enkhuizen, Netherlands. The plant history was taken on 30 week old plants, grown in the open field in Enkhuizen, The Netherlands with day temperatures ranging from 16 and 30° Celsius, and night temperatures ranging from 10 to 16° Celsius. Color references are primarily to the R.H.S. Color Chart of The Royal Horticultural Society of London.

### THE PLANT

Classification — botanical: Brachyscome multifida.
Parentage:
   Female parent.—Proprietary selection of Brachyscome multifida, identified as ‘A317-1,’ not patented.
   Male parent.—An unknown seedling or variety of Brachyscome multifida.
Propagation:
   Type cuttings.—Terminal vegetative cuttings.
   Time to intiated roots.—10-12 days at air temperature of 21 degrees C.
   Time to develop roots.—15-20 days at air temperature of 18 degrees C.
   Root description.—Fibrous, relatively fine, white in color.
   Rooting description.—Freely branching, dense.
Plant description:
   Growth habit.—Upright and rounded shape.
   Plant height.—20cm.
   Vigour.—Compact, medium vigor.
   Spreading area of plant.—Approximately 42 cm.
   Strength.—Very strong.
   Branching character.—Freely branching, plants do not require pinching. Commonly develops approximately 15 lateral branches.
   Crop time.—About 10weeks are required to produce a finished flowering plant in a 10.5 cm container from a good developed cutting.
Lateral branches:
   Length.—20 cm.
   Diameter.—Up to 4 mm.
   Shape.—Round.
   Anthocyanin pigmentation.—Absent.
   Texture.—Young stems: pubescent; older stems: smooth.
   Length of internode.—Up to 24 mm.
   Color.—RHS 146D.
The foliage:
   Arrangement.—Alternate, single.
   Shape of leaf.—2 pinnate, fern-like leaves, fully incised.
   Leaf.—Apex: Acute Base: Truncate.
   Margin.—5-7 lobed with minute teeth.
   Length.—0.5-2.5 cm.
   Width.—Up to 1.4 cm.
   Texture.—Smooth.
   Color.—Upper side: RHS 137A Lower side: RHS 137C.
   Venation.—Pattern: Pinnate Color: RHS 137A.
Inflorescence description:
   Appearance.—Disc and ray florets develop acropetally on a capitulum. Inflorescences held upright and outwardly on terminal and axillary peduncles. Inflorescences positioned perpendicular to the peduncles.
   Flowering response.—Under natural conditions, plants flower from spring to early fall in N-W Europe; plants flower freely continuous during this period. Flowers last up to 14 days.
Flower bud:
   Peduncle.—Shape: Rounded Length: 4-7 cm Diameter: 1-2 mm Strength: Strong, flexible Aspect: Erect Texture: Smooth Color: RHS 144A.
   Inflorescence bud.—Height: 3 mm Diameter: 3-4 mm Shape: Ovoid Color: RHS 144A.
Inflorescence:
   Size.—Diameter: 15 mm depth: 4 mm.
   Diameter of disc.—4 mm.
   Form.—Semi double, composite, persistent inflorescences.
Ray florets:
   Quantity.—About 2 whorls, totally 17-26 ray florets.
   Aspect.—Flat to slightly reflexed.
   Shape.—Ensiform.
   Apex.—Acute.
   Base.—Attenuate.
   Texture.—Very light, pubescent.
   Margin.—Entire.
   Length.—6mm.
   Width.—1 mm.
   Color.—Upper: Young flowers: RHS N82B, old flowers RHS N82D Lower: Lighter than RHS N82D, stripes RHS N81A.
   Fragrance.—A light scent of citrus fruit.
Disc florets:
   Arrangement.—Massed at the center of the inflorescence.
   Shape of disc florets.—Oblong.
   Apex.—Acute.
   Base.—Fused.
   Margin.—Scarious.
   Aspect.—Concave.
   Texture.—Some hairs.
   Color.—Immature: RHS 6B Mature: RHS 6A.
   Disc floret.—Diamater: 0.4 mm Length: 4 mm.
   Quantity per inflorescence.—±25 .
   Sepals.—Shape: Ligulate with rounded apex; base fused Number: ±14 Length: 1.5 mm Width: 0.5-1 mm Color: RHS 144A.
Androecium:
   Present only on disc florets.—
   Nr of stamens.—Five per floret.
   Anther shape.—Linear, oblong.
   Amount of pollen.—Moderate to abundant
   Pollen color.—Yellow, RHS 25A.
Gynoecium:
   Present on both ray and disc florets.
   Pistil number.—one per floret.
   Style.—Length: 2 mm Color: RHS 157C.
   Stigma.—Color: Yellow, RHS 9B Shape: Bilobed.
Seed/fruit: Seed and fruit production has not been observed to date.
Disease resistance: Neither susceptibility nor resistance to fungal, bacterial or viral pathogens has been noted.
Temperature/weather tolerance: Plants of the new Brachyscome have been observed to be tolerant to rain, wind and to temperatures from 10C. to 30° C.

What is claimed is:

1. A new and distinct variety of Brachyscome plant, substantially as illustrated and described herein.
